(19) United States
(12) Patent Application Publication
(10) Pub. No.: US 2015/0359499 A1
(43) Pub. Date: Dec. 17, 2015

(54) INTRAOPERATIVE DETECTION OF TUMOR RESIDUES USING BETA-RADIATION AND CORRESPONDING PROBES

(71) Applicants: UNIVERSITA' DEGLI STUDI DI ROMA "LA SAPIENZA", Roma (RM) (IT); ISTITUTO NAZIONALE DI FISICA NUCLEARE, Frascati, Roma (IT); MUSEO STORICO DELLA FISICA E CENTRO STUDI E RICHERCHE "E. FERMI", Roma (RM) (IT)

(72) Inventors: Riccardo FACCINI, Roma (RM) (IT); Radio BELLINI, Roma (RM) (IT); Francesco COLLAMATI, Roma (RM) (IT); Antonio DI DOMENICO, Roma (RM) (IT); Erika DE LUCIA, Roma (RM) (IT); Fernando FERRONI, Roma (RM) (IT); Salvatore FIORE, Roma (RM) (IT); Michela MARAFINI, Roma (RM) (IT); Ilaria MATTEI, Roma (RM) (IT); Vincenzo PATERA, Roma (RM) (IT); Luca PIERSANTI, Roma (RM) (IT); Alessio SARTI, Roma (RM) (IT); Adalberto SCIUBBA, Roma (RM) (IT); Cecilia VOENA, Roma (RM) (IT)

(21) Appl. No.: 14/763,328

(22) PCT Filed: Jan. 29, 2014
(86) PCT No.: PCT/IT2014/000025

§ 371 Date: Jul. 24, 2015

(30) Foreign Application Priority Data
Jan. 29, 2013 (IT) RM2013A000050

(51) Int. Cl. A61B 6/00; A61K 51/08; A61K 51/04; A61K 51/10; A61K 51/12

(52) U.S. Cl. CPC A61B 6/481; A61B 6/4258; A61B 6/4216; A61B 6/4405; A61K 51/1096; A61K 51/1251; A61K 51/0489; A61K 51/088; A61K 51/04

(57) ABSTRACT

The invention relates to the use of β⁻ emitting radiolabeled tracers for administration to a patient prior to radio-guided surgery, and to the corresponding probes designed to intraoperatively detect β⁻ decays from cancerous tissues so as to locate even small cancerous remnants still present after resection of the main cancerous lesions. The β⁻ emitting radiolabeled tracer is labeled with a radioisotope undergoing exclusively β⁻ decays or a radioisotope undergoing β⁻ decays and having no more than 10-11% of γ rays decays. The corresponding probe has an extension direction along a longitudinal axis and has one or more blocks of scintillating material, each one having a main extension parallel to the longitudinal axis and transversal dimensions smaller than 3 mm, with each of the blocks being partially shielded by a material that is inactive with respect to β⁻ radiation.

### FIELD OF THE INVENTION

The present invention concerns a method for the intraoperative detection of tumor residues using β⁻ radiation, radiopharmaceuticals labeled with β⁻ emitters for use in such method and the corresponding β⁻ probes. More specifically, the invention relates to the use of β⁻ emitting radiolabeled tracers for administration to a patient prior to radio-guided surgery, and to the corresponding probes designed to intraoperatively detect β⁻ decays from cancerous tissues so as to effectively locate small cancerous remnants still present after resection of the main cancerous lesions.

### BACKGROUND OF THE INVENTION

In oncologic surgery, the complete resection of a tumor is a necessary condition to avoid relapses and thus to achieve the recovery of the patient. The knowledge of the boundaries of the cancerous lesions as well as the identification of possible lymph-nodes, which may well be quite distant from the main tumor but have been contaminated by it, are critical facts for the success of a cancer resection surgery.

The surgeon can be led to this purpose by images collected before the surgery. However, when the resection of non-palpable remnants is to be performed, this support is normally not effective because the organs have moved meanwhile, and because the spatial resolution of the available images does not allow to detect small remnants.

It is extremely important, therefore, to develop techniques which may allow the surgeon to locate cancerous lesions during the surgery. To this end, current research is focused on the following techniques:
1) Use of luminescent materials, absorbed exclusively by some tumors, whose emitted light can be detected by infrared filters. This technique has the disadvantage that it may be only applied to particular tumors, and that it alters the surgeons' operating conditions.
2) Use of intraoperative Nuclear Magnetic Resonance. This technique is very expensive and requires interruption of the surgery for a long time span. Moreover, the surgery conditions (presence of blood, equipment, etc.) can alter the image.
3) Use of tracers containing nuclides which decay producing beta⁺ (β⁺) and/or or gamma (γ) radiation. Radioactive tracers normally used for diagnostic imaging outside the surgery framework, i.e., SPECT (Single Photon Emission Computed Tomography) and PET (Positron Emission Tomography) scans, can also be applied in the surgery framework, by using local probes which detect the positrons (i.e., β⁺ particles) or the photons emitted by their annihilation. These techniques have the disadvantage of exposing the medical personnel to the radiation emitted by the patient, owing to the fact that the emitted gamma radiation is penetrating.

As it is known, gamma radiation is high-energy electromagnetic radiation (i.e. photons) emanating from atomic nuclei, while beta radiation is the result of the corresponding weak decay of nucleons inside atomic nuclei. These charged particles can be conventional negatively charged electrons (“beta minus”) or, in the case of “beta plus” decay, positively charged electrons, that is, positrons. When a positron, being the antimatter counterpart of the ordinary electron, encounters an electron, mutual annihilation occurs with the complete conversion of all mass into energy. The energy is released in the form of two photons of similar energy traveling 180° apart, and these easily detected gamma photons form the basis of PET imaging. Thus, the image-generating photons in PET come from within the patient, rather than coming from an external source and traveling through the patient to form an image, as X-ray photons in computerized tomography. Developing tumors, with their relatively high metabolism, tend to accumulate the administered tracer, which in the case of PET is tagged with a positron-emitting radioisotope such as fluorine-18 (¹⁸F), providing the selectivity useful in oncology.

The third technique mentioned above, more specifically the radio-guided surgery (RGS), was first developed some 60 years ago, as a surgical technique aimed at enabling the surgeon to perform complete lesion resections, while minimizing the amount of healthy tissue removed (see, for instance, Mariani G, Giuliano A E, Strauss H W Edts, “Radioguided Surgery: A Comprehensive Team Approach” Springer (2006)).

The basic idea is to administer to the patient, before surgery, a radiolabeled tracer that is preferentially taken up by the tumor and to exploit, during surgery, a specific probe system to detect the emission released by the targeted tumor cells in real time (see, for instance, Hoffman E J et al. “Intraoperative probes and imaging probes”, Eur J Nucl Med (1999) 26:913; Povoski S P et al. “A comprehensive overview of radioguided surgery using gamma detection probe technology”, World Journal of Surgical Oncology (2009) 7:11; Tsuchimochi M and Hayamaand K, “Intraoperative gamma cameras for radioguided surgery: Technical characteristics, performance parameters, and clinical applications” Physica Medica (2013) 29, 126).

After the mass removal, the surgeon explores the lesion with the radiation detection probe and looks for tumor remnants, difficult to identify by the naked eye. For pathologies like breast, kidney and stomach tumors the risk of recidivism comes also from infected lymph-nodes: in such cases the probe helps during surgery by identifying which lymphatic chain is affected and therefore by allowing to remove all and only the infected lymph-nodes. Hence, the impact of RGS on the surgical management of cancer patients includes providing vital and real-time information to the surgeon regarding the location and extent of the disease, as well as allowing the assessment of surgical resection margins.

Some current clinical applications of RGS are: radio-immuno-guided surgery (RIGS) for colon cancer, complete sentinel-node mapping for malignant melanoma and breast cancer, and detection of parathyroid adenoma and bone tumors (such as osteoid osteoma).

In general, established methods make use of a gamma (γ) radiation detection probe, but other radiation detection devices, exploiting beta⁺ (β⁺) decaying tracers, are under development (see, e.g., Bogalhas, F. et al. “Development of a positron probe for localization and excision of brain tumours during surgery.” Phys. Med. Biol. 54 443-4453 (2009)). The β⁺ decays emit positrons which can be detected directly. Nonetheless, interacting with the electrons in the body, they annihilate and produce gamma rays, so for many respects the two techniques share the same characteristics.

A limit to the applicability of the RGS in both the above techniques comes from the high penetration power of the γ radiation, i.e. from the fact that γ radiation can traverse large amounts of tissue. This has the following consequences:
a possible uptake of the tracer in nearby healthy tissue would represent a non-negligible background, sometimes preventing the applicability of the technique. Thus, the current use of RGS is quite limited, in that the following uses would be prevented:
   in brain tumors, because of the large uptake of radiotracers from the healthy brain when β⁺ emitting tracers, such as the common radiolabeled tracer ¹⁸F-FDG (fluorodioxyglucose), are administered;
   in abdominal tumors, because of the presence of large amounts of traces in the bladder, kidneys, liver, etc.;
   in pediatric tumors, where the distances between organs are smaller.
the solution proposed by the prior art in order to deal with the presence of the gamma background is to insert into the device additional channels to measure such background, (see, e.g., Hickernell T S et al “Dual detector Probe for surgical Tumor Staging”, The Journal of Nuclear Medicine, Vol. 29:6, (1988) 1101-1106; Bonzom, S. et al. “An Intraoperative Beta Probe Dedicated to Glioma Surgery: Design and Feasibility Study” IEEE Trans. Nucl. Sci. 54(1) 30-41 (2007)), which makes the corresponding probes cumbersome and difficult to manage.
the activity of radiotracer that needs to be administered to achieve the required rapidity in the answer in cases different from those presently in use would be large, thereby resulting in an increase of the exposure of both patient and medical staff. The latter circumstance would limit the number of patients per year that could be treated with RGS by each member of the medical staff.

It is to be noted, incidentally, that the above problems do not apply to the existing applications of RGS, as said applications concentrate on evaluating contamination of lymph-nodes of known position and far enough from up-taking organs.

Other known devices and methods which exploit the radiation emitted by radiopharmaceuticals suitable for PET scans in connection with intraoperative probes designed to detect both beta⁺ (i.e., positrons) and gamma radiation, and which include specific solutions to take into account the presence of gamma radiation are disclosed, e.g., by the U.S. Pat. No. 6,149,593 (in the name of Gonzalez-Lepera, assigned to The Trustees of the University of Pennsylvania) and by the U.S. Pat. No. 6,643,538 (in the name of Majewski et al., assigned to Southeastern University Research Assn.).

In view of the foregoing prior art, the present invention is aimed at providing intraoperative detection methods and probes which would allow to extend the applicability of the radioguided surgery, by overcoming the above mentioned problems.

### SUMMARY OF THE INVENTION

In the frame of the studies connected with the present invention, it has been considered that by using the β⁻ radiation sources instead of the known gamma or β⁺ radiation sources, it would be possible to achieve the main advantage of using a non-penetrating radiation (i.e., electrons) which does not escape the patient and does not produce gamma rays. Actually, while β⁺ radiation produces a non penetrating positron which then annihilates producing gamma radiation, β⁻ radiation is limited to non-penetrating electrons.

According to the invention, it has been considered that the β⁻ radiation penetrates only a few millimeters in flesh, and this results in the following advantages:
reduced background from nearby up-taking organs and therefore:
   applicability to new cases;
   a more compact probe;
   smaller activity to administer and therefore lower dose to the medical personnel. It should be noted that it is not obvious that the patient gets a small dose, because this depends on the lifetime of the radiotracer. For a given duration of the surgical intervention, instead, the dose is for sure reduced if the activity decreases.
better directionality/spatial resolution.

Thus, the present invention is based on the consideration that whenever there is an opportunity to deliver the same tracer with a β⁺ or a β⁻ radionuclide, it is always convenient to choose the β⁻ radionuclide for the intraoperative application. Assuming to use the same device (or a device with similar performances) to detect the beta radiation in both cases, for a given activity of the tumor residue that needs to be detected the signal coming from the beta particles would be the same. But if the administered tracer decays β⁺ there is an additional background due to gammas, which would not otherwise be present with a β⁻ radiotracer.

Even if the user were able to subtract the background with infinite precision, the probability of a false positive would always, by definition, be worse in the case of a β⁺ tracer, because of Poisson fluctuations of the background. This means that the activity to be administered to achieve a specific rate of false positives and false negatives would be, by definition, larger in the case of a β⁺ tracer.

Finally, while the two-channel system proposed by the prior art mentioned above can properly subtract the background, the amount of such background, and therefore its fluctuations, will vary from patient to patient, thus making the sensitivity of such method difficult to predict.

In view of the foregoing, the choice of using the β⁻ radiation is advantageous for RGS in a number of different specific fields, and is applicable in a number of clinical cases of interest, once the appropriate radiotracers suitable to carry β⁻ emitting radioisotopes to the tumor of interest are selected.

According to the invention, the radioisotope for use in the intraoperative detection method proposed must be a pure or “almost pure” β⁻ emitter, namely, it must either be a pure β⁻ emitting radionuclide such as yttrium-90 (⁹⁰Y), strontium-89 (⁸⁹Sr), scandium-49 (⁴⁹Sc), silicon-31 (³¹Si) and zinc-69 (⁶⁹Zn), or a radionuclide which also emits, albeit to a lesser extent, gamma radiation. In the latter case, the γ radiation is emitted with an abundance of no more than 10-11%, as is the case of iodine-131 (¹³¹I), and, more preferably, no more than 5%, as is the case, e.g., of potassium-42 (⁴²K) and samarium-153 (¹⁵³Sm).

Although β⁻ emitting isotopes and radiopharmaceuticals containing them have not been used so far in radioguided surgery, this class of nuclides and related compounds is already used in some cancer therapies (i.e. radiotherapy), and specific pharmacological carriers have been developed. Specifically, in nuclear/metabolic therapy β⁻ emitting isotopes are administered systemically, while in brachytherapy they are directly deposited on the tumor. Also in this case the short range of electrons is exploited, since it allows the bulk of the dose release to be confined in the tumor.

An example of molecules designed to carry radioisotopes which decay and produce β⁻ radiation, in particular yttrium-90, is disclosed in the International patent application no. WO 2012/089336 (Università degli Studi di Genova and Istituto Europeo di Oncologia). Such document concerns a conjugate of human albumin and 2-(4-isothiocyanatobenzyl)-1,4,7,10-tetraazacyclododecane-1,4,7,10-tetraacetic acid (p-SCN-Bn-DOTA), which may be used, upon being made into a complex with a suitable radionuclide, either in radio-metabolic therapy or as a diagnostic tool, depending on the radionuclide employed. The radionuclides disclosed for use with the mentioned radiopharmaceutical are either β emitters or β and γ emitters, and when the pure β⁻-emitting isotope ⁹⁰Y is mentioned, the document acknowledges that said radionuclide, lacking γ emissions, cannot be used in imaging or dosimetry, and limits its utility to the treatment (radiotherapy).

The present invention, therefore, proposes to use suitable β⁻ emitting tracers, wherein the tracer compound is selected according to the specific type of tumor to be targeted and is labeled with a pure or almost pure β⁻ emitter, as radiolabeled tracers to be administered to a patient before surgery, in order to obtain the preferential uptake of the radiotracer in the location of any residual cancerous tissue to be excised.

In combination with the proposed use of β⁻ emitting tracers before surgery, the present invention proposes to use suitably designed detection probes for the intraoperative detection of the β⁻ radiation, so as to properly locate any small remnants of cancerous tissue while overcoming the drawbacks shown by the use of the intraoperative probes of the prior art.

According to the invention, it is advantageous to use a tracer that is labeled with radioisotopes undergoing exclusively β⁻ decays, i.e. emitting only electrons that traverse at most few centimeters of matter. A suitable probe is able to identify in few seconds millimetric remnants by injecting in the patient very little doses of radiotracer, and therefore with minimum radioactive impact on the patient and health-care personnel.

The use of β⁻ radiation allows to design probes that are particularly compact and handy. However, since the use of other non-pure β⁻ emitting radionuclides (such as for example iodine-131, which is used in radio-metabolic treatment of the thyroid) is conceivable, and is expected to result in similar advantages over the use of gamma-emitting radionuclide, the solution proposed by the present invention encompasses this case as well.

It is to be noted that the probe device proposed according to the invention appears to be remarkably compact, and that in the embodiments thereof having more than one active element, all such active elements have one and the same function, as opposed to the known probes of the prior art.

### BRIEF DESCRIPTION OF THE DRAWINGS

The specific features of the invention, as well as the advantages of the same and the corresponding operation modes, will be apparent with reference to the detailed description presented hereafter. The latter are illustrated by way of example in the enclosed drawings, wherein:

FIG. 1 is a schematic cross-sectional view of a first embodiment of the probe according to the invention;

FIG. 2 is a schematic cross-sectional view of a second embodiment of the probe according to the invention;

FIG. 3 is a schematic cross-sectional view of a third embodiment of the probe according to the invention;

FIG. 4 shows the external appearance of a prototype of the probe according to the invention; and

FIG. 5 is a schematic cross-sectional view of a fourth embodiment of the probe according to the invention.

### DETAILED DESCRIPTION OF THE INVENTION

The present invention specifically provides a β⁻ emitting radiolabeled tracer for use as radioactive tracer to be administered to a patient before radio-guided surgery, wherein the tracer is labeled with a radioisotope undergoing exclusively β⁻ decays—i.e. a pure β⁻ emitter—or a radioisotope undergoing β⁻ decays and having no more than 10-11% of γ rays decays. Preferably, the abundance of γ decays of the “almost pure” pure β⁻ emitter is <5%, and more preferably it is <2%.

Preferably, as pointed out before, the radioisotope for use according to the invention is selected from the following group of pure β⁻ emitters: yttrium-90 (⁹⁰Y), strontium-89 (⁸⁹Sr), scandium-49 (⁴⁹Sc), silicon-31 (³¹Si) and zinc-69 (⁶⁹Zn). In the alternative, the radioisotope may be selected from the following group of “almost pure” β⁻ emitters: potassium-42 (⁴²K), samarium-153 (¹⁵³Sm) and iodine-131 (¹³¹I). These radioisotopes are listed in the following Table 1, where the respective half-lives and the purity of their β⁻ decays are reported.

TABLE 1

| Isotope | ½-life | | Pure? |
|---|---|---|---|
| ⁹⁰Y | 64 | h | Y |
| ⁸⁹Sr | 50 | d | Y |
| ⁴⁹Sc | 1 | hr | Y |
| ³¹Si | 2 | hr | Y |
| ⁶⁹Zn | 1 | hr | Y |
| ⁴²K | 12 | h | N |
| ¹⁵³Sm | 46 | h | N |
| ¹³¹I | 8 | d | N |

With reference to the most interesting medical applications of the radio-guided surgery (RGS) method using β⁻ radiation of the invention, the following issues should be noted. Firstly, as pointed out before, a complete removal of tumor is particularly critical for brain tumors, where a relapse is quite dangerous and where other RGS techniques using β⁺ and γ radiation are limited by the high uptake of the brain.

As a second issue, particular attention should be paid to the complete resection of the main tumor and of infected lymph-nodes in the case of pediatric tumors, where life expectancy is long. Such tumors are typically abdominal, and therefore probe signals are blinded by background from kidneys, liver and bladder. As a third issue, there are some abdominal tumors in adults, like non-palpable metastases in liver and insulinoma, that would profit from the proposed RGS using β⁻ radiation.

In this connection, it is to be noted that:
in the case of radionuclide therapy the administered activity is several orders of magnitude higher than the activity of the gamma emitters usually administered for imaging, and equally higher than the activity that this invention would use;
in the case of therapy the requirement of the β⁻ emitter being pure is looser: the gamma radiation would largely escape the patient and keeping the patient isolated is a good enough protection to the medical personnel, while isolation that is not possible in intraoperative uses;
after a patient is administered a therapeutic activity of β⁻ emitter there is copious bremsstrahlung emission due to the interaction of the electrons with the cells. Such radiation is often used for diagnostic use, since it is intense enough that the gamma cameras used for SPECT give images with acceptable resolution. This diagnostic use is completely different from the one proposed by this invention, because the β⁻ radiation is not detected directly, being the use intraoperative, and therefore the goals are completely different.

Commonly used radiotracers suitable for use in the method of the present invention can be selected from those already in use for the in the field of therapy. Some of them are shown in the following Table 2 (taken from the review: Targeted Radionuclide Therapy by Devrim Ersahin, Indukala Doddamane and David Cheng, Cancers (2011), 3(4), 3838-3855).

TABLE 2

| Radio pharmaceutical | Targeting mechanism | Indications |
|---|---|---|
| I-131 as iodide | Thyroid hormone synthesis | Differentiated thyroid carcinomas |
| 1-131 Tositumomab | CD20 Antigen binding | Non-Hodgkin's lymphoma |
| Y-50 Ibritumomab | CD20 Antigen binding | Non-Hodgkin's lymphoma |
| tiuxetan | | |
| Y-90 mircrospheres | Intravascular trapping | Liver metastasis Hepatocellular |
| | | carcinoma |
| Sr-89 chloride | Calcium analogue | Bone pain palliation |
| Sm-153 EDTMP | Chemoadsorption | Bone pain palliation |
| Y-90 Octreotide | Somatostatin receptor binding | Neuroendocrine tumors |
| I-131 MIBG | Active transport into | Neuroblastoma |
| | neuroendocrine cells and | Pheochromacytoma |
| | intracellular storage | Carcinoid |
| | | Paraganglioma |
| | | Medullary thyroid carcinoma |

According to some preferred embodiments of the of the present invention, the radiolabeled β⁻ emitter may comprise, in particular, a tracer compound selected from the group consisting of: a salt of the corresponding radionuclide, a radiolabeled antibody, a radiolabeled peptide receptor, and a radiolabeled meta-iodobenzylguanidine (MIBG).

According to some further preferred embodiments of the invention, the tracer compound may be selected from a group of known radiolabeled peptide receptors including [Y-90-dodecanetetraacetic acid (DOTA), Tyr3]-octreotide (i.e. Y-90-DOTATOC), a conjugate of human albumin and 2-(4-isothiocyanatobenzyl)-1,4,7,10-tetraazacyclododecane-1,4,7,10-tetraacetic acid (p-SCN-Bn-DOTA) labeled with Y-90, and [Y-90-DOTA]-lanreotide.

The intraoperative method of detection of tumor residues using β⁻ radiation according to the invention involves administering to the patient, at a suitable time before surgery, a β⁻ emitting radiolabeled tracer according to the invention, as defined above, and then, during the surgery, using a specifically designed β⁻ detection probe to intraoperatively detect any β⁻ emitting tumor residues to be excised. Thus, the invention also concerns a β⁻ surgical probe as described below.

According to this further aspect thereof, the invention specifically provides a β⁻ detection probe for the intraoperative recognition of tumor residues, having an extension direction along a longitudinal axis and comprising one or more blocks of scintillating material each one having a main extension parallel to said longitudinal axis and transversal dimensions smaller than 3 mm, each of said one or more blocks being partially shielded by a material that is inactive with respect to β⁻ radiation, the partial shielding being such that at least a surface portion of each block is active with respect to β⁻ radiation, said surface portion being connectable to a light detector.

By the term “surface portion” an area of the surface of a block has to be understood; specifically, in case of parallelepiped blocks one single face has to be understood, while in case of a cylinder one of the bases or a portion of the lateral surface. In such a way, a directionality of the detection performed by said at least one block is obtained.

Preferably, according to the invention, the extension along said longitudinal axis of the probe is comprised, for ease of handling, between 10 and 20 cm, while the functional elements of the probe may be limited to a much shorter stretch of said length, said one or more blocks of scintillating material being located in or closer to a tip section of the probe. The transversal dimensions are smaller than or equal to 2 cm.

Preferably, according to the invention, at least a portion of the probe other than the tip section is flexible.

According to a preferred embodiment of the invention, the density of said one or more blocks of scintillating material is smaller than 5 g/cm³, and said inactive material shields the gamma radiation as well. The low density entails a poor sensitivity to photons.

Preferably, according to the invention, the probe comprises one single block with one only unshielded surface, that therefore constitutes the tip of the probe. In the present disclosure, by the term “unshielded” or “not shielded” it is not to be understood that the face is not coated by any material, but that the possible coating does not sensibly lower the sensitivity of the scintillating material.

According to another embodiment of the invention, the claimed probe comprises:
a first parallelepipedal block of scintillating material, comprising four larger faces and two smaller faces, one of the smaller faces being unshielded, and
four further parallelepipedal blocks of scintillating material having corresponding larger faces parallel to said longitudinal axis, said four further parallelepipedal blocks being placed side-by-side to said first block along said four faces thereof, each one of the four further parallelepipedal blocks presenting the face placed side-by side to the first parallelepipedal block unshielded. It should be noted that in the present embodiment, differently from the probes of the prior art, all of the active elements have the same function, namely to detect β⁻ radiation.

According to the invention, the probe may alternatively comprise a plurality of blocks of scintillating material, each being a parallelepiped and each presenting a shielded face on a same plane, the whole of unshielded faces constituting the tip of the probe. Also in the instant embodiment, differently from the probes of the prior art, all of the active elements have the same function, namely to detect β⁻ radiation.

According to another embodiment of the invention, the probe consists of the following elements:
a hollow scintillator cylinder with internal diameter of 1-3 mm and external diameter of 4-7 mm, longitudinal extension 3-4 mm, as an only piece or in equal sectors each connectable to an optical reading system;
an internal scintillator cylinder with diameter of 1-3 mm, longitudinal extension substantially equal to said hollow scintillator cylinder, connectable to a optical reading system and inserted in the hollow scintillator cylinder without protruding from it;
a metal or plastic material shield consisting of a circular corona, with dimensions substantially equal to the section of said hollow scintillator cylinder and integral with it, with maximum height 3 mm, such as to shield from the β⁻ radiation said hollow scintillator cylinder but not the internal scintillator cylinder. The fact that each sector can be connected to a detection system is because in such a way one is able to evaluate whether an electron comes laterally, and more exactly from which side.

Preferably, according to the invention, said scintillating material is p-terphenyl or CsI(Tl) or other scintillating material, selected from materials with light yield larger than 15,000 photons/MeV and attenuation length smaller than 50 cm.

According to the invention, said inactive material is preferably a metallic material or a plastic material, in particular PVC.

The present invention further specifically concerns a device for the detection of β⁻ radiation in an intraoperative framework, including a probe as disclosed above and a light-detecting device.

Preferably, according to the invention, said light detecting device is a photomultiplier or other photodetector connected to said probe by scintillating fiber or a solid state photodetector. The scintillation light can be read directly by a solid-state detector (SiPM, APD or G-APD) or transported by fibers to a photomultiplier or other photosensitive device.

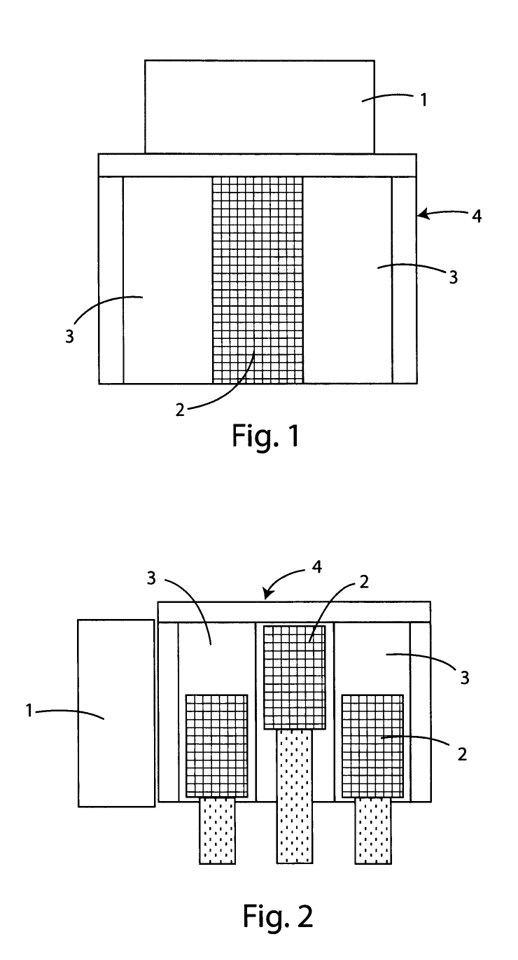

The various embodiments of the probe according to the invention are further described in detail with reference to the attached drawings. With reference to FIG. 1, a first embodiment of the claimed probe includes a probe tip (4) composed by one only scintillator (2), for example cubic with side of 2 mm, preferably made of p-terphenyl (PTERP) (where the scintillator material is shown by a square-grid hatch pattern in the figure). The scintillator (2) is laterally shielded by corresponding 2 mm of plastic or metallic non-active material (3) (for example PVC). This probe is optimized for the exact individuation of minimum remnants of a tumor (schematically shown by (1)) with high directionality. The remnant must be located directly in front of the tip of the probe tip (4).

With reference to FIG. 2, where the same reference numbers show corresponding elements, a second embodiment of the probe has a geometry similar to the previous one, wherein however a part of the non-active material (3) is replaced by scintillating material (2), so as to be sensitive also to the radiation coming from the side. This figure also schematically shows the connections of each block of scintillating material (3) to a light detecting device (shown by a dotted hatch pattern in the figure), as described above. This type of probe is useful when it is suspected that the tumor remnant is hidden by other structures between which the probe is to be inserted.

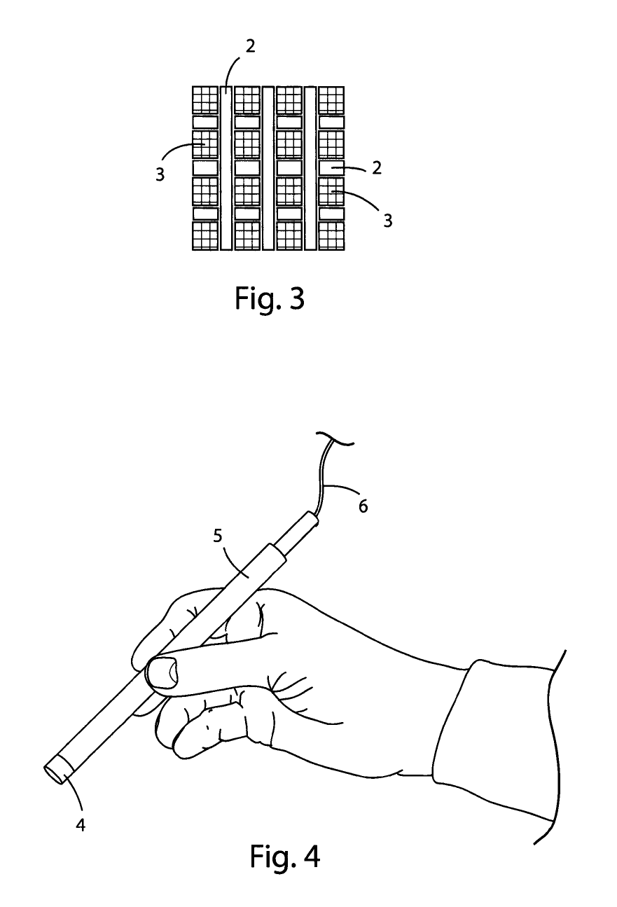

With reference to FIG. 3, where the same reference numbers show corresponding elements as in the other figures, a third embodiment of the probe is schematically shown. The probe has an array of detectors which allows to evaluate the presence of remnants in a wider field, in order to avoid both an excessive duration of the verification in the operating theatre and the accidental missing of a remnant. In the RGS according to this invention, this kind of array probe can be used first, followed by the above described “point-like” probes.

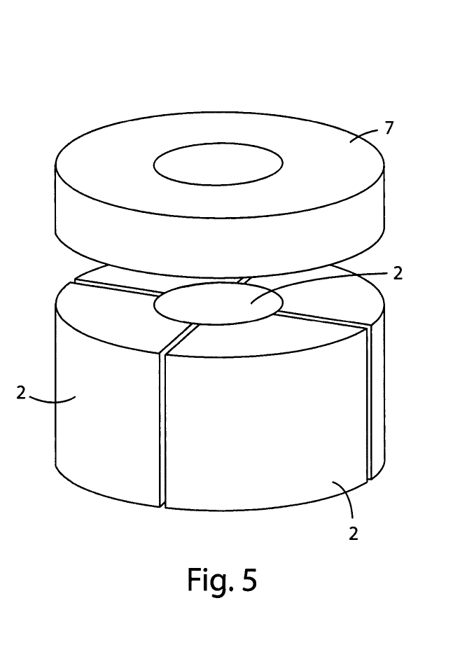

With reference to FIGS. 4 and 5, where the same reference numbers show corresponding elements, a fourth embodiment of the probe tip (4) includes:

a hollow scintillator cylinder with internal diameter of 1-3 mm and an external diameter of 4-7 mm, longitudinal extension of 3-4 mm, as an only piece or as blocks (2) in the form of equal sectors, each block being connectable to a system of optical reading;

an internal scintillator cylinder (2) with diameter of 1-3 mm and longitudinal extension substantially equal to that of said hollow scintillator cylinder, the internal scintillator cylinder being connectable to an optical reading system and inserted in the hollow cylinder without protruding from it;

a metallic or plastic material shield consisting of a circular corona (7), with dimensions equal to the section of said hollow scintillator cylinder and integral with it, with maximum height of 3 mm, such as to shield the front β⁻ radiation of all the elements of said hollow scintillator cylinder but not of the central one.

The probe shown in its entirety in FIG. 4 also includes a main body (5) which is designed to provide a comfortable handling of the probe, and which may also be made flexible in at least a portion of its length, and a connecting element (6) for transporting the scintillation light or the corresponding electric signal to a photosensitive device or directly to an electronic device providing the corresponding acoustic and visual signaling system.

For each one of the above embodiments the following specifications hold:
the scintillator (2) can be:
   p-terphenyl (PTERP). Only recently it has been found that this scintillator, which produces much light upon the passage of the particles (high “light yield”) but allows this light to come out in a small proportion (short attenuation length), can be used in applications wherein the detectors are a few millimeters long (Angelone M. et al., “Properties of para-terphenyl as detector for α, β, and γ radiation”, arXiv:1305.0442, submitted to T.N.S.). The advantage of this solution is that the possible presence of γ radiation (in a quantity that can be associated to a β decay, surely much less than the quantity generated in β⁺ decays) does not jeopardize the measurement;
   CsI(Tl), a scintillator with most high light yield, which allows to increase the sensitivity to the electrons (and therefore to decrease the dose of radiopharmaceutical to be injected). It is however very sensitive also to γ rays;
   possible other scintillating materials that have been discarded in the past because of their short attenuation length, but that would be functional to this application.
The scintillating light produced by the passage of the electrons
   can be transported by scintillating fiber and read by a photomultiplier or other photosensitive device;
   can be read directly on the crystal with a solid-state photo-detector (for example an avalanche photodiode (APD) or Geiger-mode APD) with a dedicated miniaturized electronics.

In the frame of the experimentation connected to the invention, the following prototypes have been studied:
a) a first one having a sensitive portion of PTERP of 1.05 mm in radius and a thickness of 1.7 mm, shielded by 2.45 mm of PVC. The reading is obtained through one single fiber which carries the signal to a PMT;

b) a second one with a sensitive portion of PTERP of 2.55 mm in radius and a thickness of 3 mm, shielded by 2.45 mm of PVC and read by four fibres connected to a PMT;
c) a third one with a sensitive portion of 5 mm in radius and thickness of 2.5 mm, and wherein instead of a PMT a SiPM (2×2 mm²) is used, placed directly on the scintillator, i.e. without employing the fiber.

For reading the probe a specifically designed electronics has been employed for the wireless transmission of the detected signal frequency to a PC or a tablet, with acoustic and visual signaling of exceeding a threshold value (to be set based on the signal emitted by the residue that it is intended to detect).

The following tests have been performed on the prototypes:
Laboratory tests with sealed sources of ⁹⁰Sr to ascertain the effective functioning of the detection chain;
Tests with ⁹⁰Y (i.e. the same substance that it is intended to use in operating theatre). To perform these tests, owing to the short average half-life of this isotope, it has been necessary to set up a suitably designed automatic system, which places the probes in close proximity to the samples of ⁹⁰Y in salt solution in a different form, so as to simulate different possible types of residue, in an automatized and reproducible way.
Ex vivo test. The first ex-vivo tests (that is, on samples taken from a patient during the surgery) are taken from patients suffering from meningioma undergoing surgery. The radiopharmaceutical is to be administered to the patients before surgery.

The tests carried out have shown that by administering a radiopharmaceutical activity equal to that administered in a PET it is possible to detect residues 1 mm thick in about 1 second of exposure with the first prototype developed. With this activity the dose received by the medical staff is negligible, while the dose received by the patient is about twice the dose of a TAC scan, but ten times lower than the dose administered for a treatment of metabolic radiotherapy. By using the other two probe prototypes it is possible to reduce the activity to be administered to the patient. In particular, the transverse dimensions of these two probes are higher, while still being within the specifications set by the surgeons. Therefore, the efficiency of the signal collection should be higher, and therefore the minimum detectable activity of the tumor is lower.

The advantages of the present invention comprise:

that the radiation utilized and emitted by the patient travels a very limited path. The radiation comes out to be therefore delimited and this allows to protect the medical staff that is in such a way exposed to a very low radiation. Moreover, the detected signals come certainly from a very close source, improving the ability to localize the residues with precision;

That the probe suitable to detect the electrons is much more compact, easily usable and economical than the existing ones, not being necessary to protect the medical staff from an intense gamma radiation (or in any case by using the p-terphenyl that allows to manage a moderate gamma radiation). This allows therefore applications in frameworks wherein the access is limited, such as for example the tumors in the neuro-surgical field;

That the radioisotope with the most suitable decay, yttrium-90, is already utilized for labeling many tracers for the purpose of metabolic therapy.

Such tracers, once administered in much smaller doses, can play a secondary role through technique proposed herein.

The present invention has been disclosed with particular reference to some specific embodiments thereof, but it should be understood that modifications and changes may be made by the persons skilled in the art without departing from the scope of the invention as defined in the appended claims.

1.-18. (canceled)

19. An intraoperative method for detecting tumor residues using β⁻ radiation, comprising:
a) administering to a patient, at a suitable time before surgery, a β⁻ emitting radiolabeled tracer labeled with a radioisotope undergoing exclusively β⁻ decays or a radioisotope undergoing β⁻ decays and having no more than 10-11% γ rays decays;
b) detecting any tumor residues by using a β⁻ detection probe having an extension direction along a longitudinal axis and comprising one or more blocks of scintillating material each one having a main extension parallel to said longitudinal axis and transversal dimensions smaller than 3 mm, each of said one or more blocks being partially shielded by a material that is inactive with respect to β⁻ radiation, the partial shielding being such that at least one surface portion of each block is active with respect to β⁻ radiation, said surface portion being connectable to a light detector, thereby evidencing any tumor residues to be excised.

20. The method of claim 19, wherein said radioisotope is selected from the group consisting of: yttrium-90, strontium-89, scandium-49, silicon-31 and zinc-69, potassium-42, samarium-153 and iodine-131.

21. The method of claim 19, wherein said radioisotope undergoing β⁻ decays has no more than 5% γ rays decays.

22. The method of claim 19, wherein said tracer compound is selected from the group consisting of: a salt of the corresponding radionuclide, a radiolabeled antibody, a radiolabeled peptide receptor, and a radiolabeled metaiodobenzylguanidine (MIBG).

23. The method of claim 22, wherein said tracer compound is selected from the group consisting of: a conjugate of human albumin and 2-(4-isothiocyanatobenzyl)-1,4,7,10-tetraazacyclododecane-1,4,7,10-tetraacetic acid (p-SCN-Bn-DOTA) labeled with Y-90 [Y-90-DOTA]-Tyr³-octreotide (Y-90-DOTATOC), and [Y-90-DOTA]-lanreotide.

24. The method of claim 19, wherein in said β⁻ detection probe the extension along said longitudinal axis of the probe is comprised between 10 and 20 cm with transversal dimensions smaller or equal to 2 cm, said one or more blocks of scintillating material being located in or closer to a tip section of said probe.

25. The method of claim 19, wherein at least one section of said β⁻ detection probe, different from said tip section, is flexible.

26. The method of claim 19, wherein the density of said one or more blocks of scintillating material is smaller than 5 g/cm³, and said inactive material shields the gamma radiation.

27. The method of claim 19, wherein said probe comprises one single block with a single unshielded surface portion, that which constitutes the tip of the probe.

28. The method of claim 19, wherein said probe comprises:
a first parallelepipedal block of scintillating material, comprising four larger faces and two smaller faces, one of the smaller faces being unshielded, and
four further parallelepipedal blocks of scintillating material having corresponding larger faces, parallel to said longitudinal axis,
said four further parallelepipedal blocks being placed side-by-side to said a first block along said four larger faces, each of the four further parallelepipedal block presenting the face placed side-by side to the a larger face unshielded.

29. The method of claim 19, wherein said blocks are parallelepipeds each presenting a shielded face on a same plane, the whole of unshielded faces constituting the tip of the probe.

30. The method of claim 19, wherein said probe comprises:
a hollow scintillator cylinder with internal diameter of 1-3 mm and external diameter of 4-7 mm, longitudinal extension 3-4 mm, as a single piece or in equal sectors each connectible to an optical reading system;
an internal scintillator cylinder with diameter of 1-3 mm, longitudinal extension substantially equal to that of said hollow scintillator cylinder, connectable to a optical reading system and inserted in the hollow scintillator cylinder without protruding from it;
a metallic or plastic material shield constituted by a circular corona, with dimensions substantially equal to the section of said hollow scintillator cylinder and integral to it, with maximum height 3 mm, such as to shield from the β⁻ radiation said hollow scintillator cylinder but not the internal scintillator cylinder.

31. The method of claim 19, wherein said scintillating material is p-terphenyl or CsI(Tl) or scintillating materials with light yield larger than 15,000 photons/MeV and attenuation length smaller than 50 cm.

32. The method of claim 19, wherein said inactive material is a metal material or a plastic material.

33. The method of claim 32, wherein the plastic material is PVC.
